(19) United States
(12) Patent Application Publication
(10) Pub. No.: US 2025/0354134 A1
(43) Pub. Date: Nov. 20, 2025

(54) GENE THERAPY SYSTEMS AND RELATED METHODS FOR TREATMENT OF HEARING LOSS

(71) Applicant: RESCUE HEARING INC., Gainesville, FL (US)

(72) Inventors: Hinrich STAECKER, Leawood, KS (US); Caesar James AYALA, Gainesville, FL (US)

(21) Appl. No.: 19/285,350

(22) Filed: Jul. 30, 2025

Related U.S. Application Data
(63) Continuation of application No. 16/783,148, filed on Feb. 5, 2020, now Pat. No. 12,391,936.
Application No. 16/783,148 is a continuation-in-part of application No. 16/726,495, filed on Dec. 24, 2019.
Application No. 16/726,495 is a continuation-in-part of application No. 16/488,103, filed on Aug. 22, 2019.
(60) Provisional application No. 62/472,790, filed on Mar. 17, 2017.
(60) Provisional application No. 62/531,522, filed on Jul. 12, 2017.

(51) Int. Cl. C12N 9/64; A61K 9/00; A61K 48/00; A61N 1/05; A61N 1/36; C12N 5/074; C12N 15/86

(52) U.S. Cl. CPC C12N 9/6421; A61K 9/0019; A61K 9/0046; A61K 48/0058; A61N 1/0541; A61N 1/36038; C12N 5/0696; C12N 15/86

(57) ABSTRACT

The present disclosure describes gene therapy systems, and related methods, useful for treating and/or preventing deafness caused by genetic mutation of the TMPRSS3 gene or the LOXHD1 gene. The compositions and methods disclosed herein use adeno-associated viral (AAV) vector gene delivery of TRMPSS3 or LOXHD1 into the inner ear to restore activity of the TMPRSS3 gene or the LOXHD1 gene, respectively, promote hair cell survival and restore hearing in patients suffering from hearing loss. As disclosed herein, the systems and methods may utilize a combination of gene therapy (e.g., molecular therapeutics) for hearing loss caused by a genetic mutation together with implantation of a cochlear implant.

### CROSS-REFERENCE TO RELATED APPLICATIONS

The present application is a continuation of U.S. application Ser. No. 16/783,148 filed on Feb. 5, 2020, which is a continuation-in-part application of U.S. application Ser. No. 16/726,495 filed on Dec. 24, 2019, which is a continuation-in-part of U.S. application Ser. No. 16/488,103 filed on Aug. 22, 2019, which is a national phase entry of PCT Application No. PCT/US2018/022873 filed on Mar. 16, 2018, which claims priority to U.S. Provisional Application No. 62/531,522 filed on Jul. 12, 2017, and U.S. Provisional Application No. 62/472,790 filed on Mar. 17, 2017, the contents of each of which are incorporated by reference herein in their entirety.

### SEQUENCE LISTING

The instant application contains a Sequence Listing which has been submitted electronically in XML file format and is hereby incorporated by reference in its entirety. Said XML copy, created on Jul. 29, 2025, is named 163991-01011_SL.xml and is 9,098 bytes in size.

### TECHNICAL FIELD

Various embodiments of the present disclosure relate generally to gene therapy systems and methods useful in the treatment and/or prevention of hearing loss. Exemplary embodiments described herein are directed to systems and related methods for preventing the further decline in a patient's hearing loss. More specifically, embodiments taught in this present disclosure relate to gene therapy systems, and related methods, useful for treating and/or preventing deafness caused by genetic mutation of the TMPRSS3 gene or the LOXHD1 gene. These systems and methods may utilize a combination of gene therapy (e.g., molecular therapeutics) for hearing loss caused by a genetic mutation together with implantation of a cochlear implant.

Hearing loss is the most common sensory deficit in humans. According to 2018 estimates on the magnitude of disabling hearing loss released by the World Health Organization (WHO), there are 466 million persons worldwide living with disabling hearing loss (432 million adults and 34 million children). The number of people with disabling hearing loss will grow to 630 million by 2030 and to over 900 million by 2050 (1 in 10 people). Over 90% of persons with disabling hearing loss (420 million) reside in the low-income regions of the world (WHO global estimates on prevalence of hearing loss, Prevention of Deafness WHO 2018).

There are currently no approved therapeutic agents for preventing or treating hearing loss or deafness. The current treatment option for those with disabling hearing loss is a cochlear implant. Cochlear implantation is a common procedure with a large associated healthcare cost, over $1,000,000 lifetime cost per patient (Mohr PE, et al.

(2000). The societal costs of severe to profound hearing loss in the United States; IntJ Technol Assess Health Care; 16(4): 1120-35).

The current demand for cochlear implants exceeds supply. The production rate of cochlear implant units manufactured is 50,000 units each year. Based on current birth rates and the incidence and prevalence of disabling hearing loss in newborns, 134,000 cochlear implants are needed annually to provide 1 cochlear implant for each afflicted child. This number increases if patients needing bilateral (2) cochlear implants are included.

The lifetime cost of a cochlear implant is prohibitive for most people and particularly for those living outside the developed nations where the majority of persons with disabling hearing loss reside. Therapeutic options are needed to provide cost effective alternatives to cochlear implants, especially for those persons living outside developed nations.

More than 50% of prelingual deafness is genetic i.e. hereditary (Centers for Disease Control and Prevention-Genetics of Hearing Loss). Hereditary hearing loss and deafness may be conductive, sensorineural, or a combination of both; syndromic (associated with malformations of the external ear or other organs or with medical problems involving other organ systems) or nonsyndromic (no associated visible abnormalities of the external ear or any related medical problems); and prelingual (before language develops) or postlingual (after language develops) (Richard JH Smith, MD, A Eliot Shearer, Michael S Hildebrand, PhD, and Guy Van Camp, PhD, Deafness and Hereditary Hearing Loss Overview, GeneReviews Initial Posting: Feb. 14, 1999; Last Revision: Jan. 9, 2014. More than 70% of hereditary hearing loss is nonsyndromic. The different gene loci for nonsyndromic deafness are designated DFN (for DeafNess). Loci are named based on mode of inheritance: DFNA (Autosomal dominant), DF B (Autosomal recessive) and DFNX (X-linked). The number following the above designations reflects the order of gene mapping and/or discovery. In the general population, the prevalence of hearing loss increases with age. This change reflects the impact of genetics and environment and the interactions between environmental triggers and an individual's genetic predisposition.

Sensorineural hearing loss (SNHL) is the most common neurodegenerative disease in humans and there are currently no approved pharmacologic interventions. SNHL can be caused by genetic disorders as well as acquired through injuries such as sound trauma and ototoxicity. Genetic diagnostics have demonstrated that there are at least 100 genes causing nonsyndromic SNHL. Recent advances in genetics and gene therapy techniques have shown that rescue of a number of recessive types of deafness is possible through gene therapy (Akil et al., 2012; Askew et al., 2015). Long term gene delivery to the inner ear has been achieved using adeno associated viral vectors (AAV) (Shu, Tao, Wang, et al., 2016). The first human clinical trial to address deafness and hearing loss using a gene therapy was (CGF166) initiated on June of 2014 and completed in December of 2019. The Principal Investigator for CGF166 was Dr. Hinrich Staecker and the trial was sponsored by Novaris. (https://clinicaltrials.gov/ct2/show/NCT02132130). An ideal disease target for translational research in this domain is a recessive genetic hearing loss that affects a defined group of cells within the inner ear and occurs postnatally after the development of speech. Prevalence of the mutation is an additional consideration.

As described herein, by carefully evaluating both the incidence of common recessive causes of hearing loss and taking into account the size of the gene, it is possible to develop a gene therapy program that has an accessible and fairly common patient population. For example, although less common than other mutations, TMPRSS3 is a fairly common cause of hearing loss that is severe enough to warrant cochlear implantation. Additionally, patients with mutations in TMPRSS3 may not respond to cochlear implantation as well as patients with other mutations (Shearer et al., 2017). This presents the opportunity of targeting TMPRSS3, or other genes such as LOXHD1, as a stand-alone therapeutic or in combination with other therapeutic agents and/or cochlear implantation to improve implant outcomes for this disorder. Table 1 (adapted from (Miyagawa, Nishio, & Usami, 2016)) demonstrates that mutations in TMPRSS3 may be the most common cause of postlingual recessive hearing loss that has a fairly limited distribution within the cochlea and, due to the size of the gene, may be built into existing AAV vectors.

TABLE 1
Incidence of different mutations in 176 adult cochlear implant patients.

| | ONSET | | | 173 | | | |
|---|---|---|---|---|---|---|---|
| MUTATION | PRE | POST | TOTAL | % OF TOTAL | GENE S2 | HAIR CELL | DOM/REC |
| [symbol] | [symbol] | [symbol] | 29 | 17% | 2347 | NO | BOTH |
| CDH23 | 6 | 7 | [symbol] | 8% | 4843 | YES | REC |
| [symbol] | 8 | 0 | 8 | 5% | 4930 | NO | REC |
| [symbol] | 3 | 4 | 7 | 4% | 7465 | YES | BOTH |
| OTOF | 4 | 0 | 4 | 2% | 6973 | YES | REC |
| MYO15A | 2 | 2 | 4 | 2% | [symbol] | YES | REC |
| [symbol] | 3 | 0 | 3 | 2% | 1504 | NO | DOM |
| TMPRSS3 | 0 | 3 | 3 | 2% | 2460 | YES | REC |
| ACTG1 | 0 | 2 | 2 | 2% | 2123 | YES | DOM |
| USHER (1 = CDH23, | 2 | 0 | 2 | 1% | [symbol] , | ? | REC |
| 1 = PCDH15) | | | | | 7042 | | |
| Mt1555A > G | 0 | 2 | 2 | 1% | NA | ? | ? |
| [symbol] | 0 | 1 | 1 | 1% | 1559 | NO | DOM |
| DFNA5 | 0 | 1 | 1 | 1% | 2276 | YES | DOM |
| [symbol] | 0 | 1 | 1 | 1% | 2882 | NO | DOM |
| [symbol] | 0 | 1 | 1 | 1% | 2915 | YES | REC |
| LOXHD1 | 1 | 0 | 1 | 1% | 3978 | YES | REC |
| [symbol] | 0 | 1 | 1 | 1% | NA | ? | ? |

[symbol]

indicates data missing or illegible when filed

The human transmembrane protease, serine 3 (TMPRSS3; also referred to as DFNB10, DFNB8, ECHOS1, TADG12; Acc: HGNC:11877) was identified by its association with both congenital (present at birth) and childhood onset autosomal recessive deafness. Mutations in the TMPRSS3 gene are associated with Autosomal Recessive Nonsyndromic Hearing Impairment type DFNB8 and 10. TMPRSS3 is a 1646 base pair gene that codes for a serine protease and is associated with DFNA 8/10 and may make up to 1-5% of patients with hearing loss undergoing cochlear implantation (Weegerink et al., 2011). Loss of function of this gene appears to result in a broad spectrum of hearing phenotypes depending on the site of the mutation. Both congenital and adult onset progressive hearing loss have been associated with the loss of this gene.

The onset of DFNB8 hearing loss is postlingual (age 10-12 years), while the onset of DFNB10 hearing loss is prelingual (congenital). This phenotypic difference reflects a genotypic difference. The DFNB8 causing variant is a splice site variant, suggesting that inefficient splicing is associated with a reduced amount of normal protein that is sufficient to prevent prelingual deafness but not sufficient to prevent eventual hearing loss. (See, Richard JH Smith, MD, et al. (2014). Genes Known to Cause Autosomal Recessive Nonsyndromic Hearing Impairment: Deafness and Hereditary Hearing Loss Overview; GeneReviews).

TMPRSS3 mutations on chromosome 21 known to cause hearing loss are

described in Table 2.

TABLE 2
TMPRSS3 MUTATIONS (CHROMOSOME 21)

| # | MUTATION NAME | REFERENCE |
|---|---|---|
| 1 | TMPRSS3, IVS4AS, | Scott H S, et al. (2001) Insertion of beta-satellite repeats |
| | G-A, −6 | identifies a transmembrane protease causing both |
| | | congenital and childhood onset autosomal recessive |
| | | deafness. Nat Genet. 27(1): 59-63. |
| 2 | TMPRSS3, 8-BP DEL, | Scott H S, et al. (2001) Insertion of beta-satellite repeats |
| | SATELLITE REPEAT | identifies a transmembrane protease causing both |
| | INS | congenital and childhood onset autosomal recessive |
| | | deafness. Nat Genet. 27(1): 59-63. |
| 3 | TMPRSS3, 1-BP DEL, | Wattenhofer M, et al. (2002) Mutations in the TMPRSS3 |
| | 207C | gene are a rare cause of childhood nonsyndromic |
| | | deafness in Caucasian patients. J Mol Med (Berl). |
| | | 80(2): 124-31. |
| 4 | c.753G > C | Masmoudi S, et al. (2001) Novel missense mutations of |
| | (p.Trp251Cys) | TMPRSS3 in two consanguineous Tunisian families |
| | | with non-syndromic autosomal recessive deafness. |
| | | Hum Mutat. 18(2): 101-8. |
| 5 | c.308A > G | Wattenhofer M, et al. (2002) Mutations in the TMPRSS3 |
| | (p.Asp103Gly) | gene are a rare cause of childhood nonsyndromic |
| | | deafness in Caucasian patients. J Mol Med (Berl). |
| | | 80(2): 124-31. |
| 6 | c.1211C > T | Wattenhofer M, et al. (2005) A novel TMPRSS3 |
| | (p.Pro404Leu) | missense mutation in a DFNB8/10 family prevents |
| | | proteolytic activation of the protein. Hum Genet. |
| | | 117(6): 528-35. |
| 7 | c.647G > T | Wattenhofer M, et al. (2005) A novel TMPRSS3 |
| | (p.Arg216Leu) | missense mutation in a DFNB8/10 family prevents |
| | | proteolytic activation of the protein. Hum Genet. |
| | | 117(6): 528-35. |
| 8 | c.579dupA | Duzkale H, et al. (2013) A systematic approach to |
| | (p.Cys194Metfs) | assessing the clinical significance of genetic variants. |
| | | Clin Genet. 84(5): 453-63. |
| 9 | c.1192C > T | Wattenhofer M, et al. (2005) A novel TMPRSS3 |
| | (p.Gln398Ter) | missense mutation in a DFNB8/10 family prevents |
| | | proteolytic activation of the protein. Hum Genet. |
| | | 117(6): 528-35. |
| 10 | c.323-6G > A | Scott H S, et al. (2001) Insertion of beta-satellite repeats |
| | | identifies a transmembrane protease causing both |
| | | congenital and childhood onset autosomal recessive |
| | | deafness. Nat Genet. 27(1): 59-63. |
| 11 | c.916G > A | Chung J, et al. (2014) A novel mutation of TMPRSS3 |
| | (p.Ala306Thr) | related to milder auditory phenotype in Korean |
| | | postlingual deafness: a possible future implication for a |
| | | personalized auditory rehabilitation. J Mol Med |
| | | (Berl). 92(6): 651-63. |
| 12 | c.208delC | Battelino S, et al. (2015) TMPRSS3 mutations in |
| | (p.His70Thrfs) | autosomal recessive nonsyndromic hearing loss._Eur |
| | | Arch Otorhinolaryngol. 273(5): 1151-4. |
| 13 | c.1276G > A | Weegerink N J, et al. (2011) Genotype-phenotype |
| | (p.Ala426Thr) | correlation in DFNB8/10 families with TMPRSS3 |
| | | mutations. J Assoc Res Otolaryngol. 12(6): 753-66. |
| 14 | c.413C > A | Eppsteiner R W, et al. (2012) Prediction of cochlear |
| | (p.Ala138Glu) | implant performance by genetic mutation: the spiral |
| | | ganglion hypothesis. Hear Res. 292(1-2): 51-8. |
| 15 | c.325C > T | Lee Y J, Park D, Kim S Y, Park W J (2003) Pathogenic |
| | (p.Arg109Trp) | mutations but not polymorphisms in congenital and |
| | | childhood onset autosomal recessive deafness disrupt |
| | | the proteolytic activity of TMPRSS3. J Med |
| | | Genet. 40(8): 629-31. |
| 16 | c.346G > A | Ganapathy A, et al. (2014) Non-syndromic hearing |
| | (p.V116M) | impairment in India: high allelic heterogeneity among |
| | | mutations in TMPRSS3, TMC1, USHIC, CDH23 and |
| | | TMIE._PLoS One. 9(1): e84773. |
| 17 | c.727G > A | Ganapathy A, et al. (2014) Non-syndromic hearing |
| | (p.G243R) | impairment in India: high allelic heterogeneity among |
| | | mutations in TMPRSS3, TMC1, USHIC, CDH23 and |
| | | TMIE._PLoS One. 9(1): e84773. |
| 18 | c.1156T > C | Ganapathy A, et al. (2014) Non-syndromic hearing |
| | (p.C386R) | impairment in India: high allelic heterogeneity among |
| | | mutations in TMPRSS3, TMC1, USHIC, CDH23 and |
| | | TMIE._PLoS One. 9(1): e84773. |

The lipoxygenase homology domains 1 gene (LOXHD1; also referred to as LH2D1, DFNB77, FLJ32670; OMIM: 613072; Acc:HGNC:26521) encodes a highly conserved protein consisting entirely of PLAT (polycystin/lipoxygenase/alpha-toxin) domains, thought to be involved in targeting proteins to the plasma membrane. Studies in mice show that this gene is expressed in the mechanosensory hair cells in the inner ear, and mutations in this gene lead to auditory defects, indicating that this gene is essential for normal hair cell function. Screening of human families segregating deafness identified a mutation in this gene which causes DFNB77, a progressive form of autosomal-recessive nonsyndromic hearing loss (ARNSHL). Alternatively spliced transcript variants encoding different isoforms have been noted for this gene.

Clinical Features of LOXHD1:
Autosomal recessive
Hearing loss, sensorineural, bilateral (milder hearing loss at low frequencies)
Congenital onset leading to cochlear implants between 7-10 years of age in Ashkenazi Jewish families
Onset by 7-8 years of age progressing to moderate-to-severe loss of mid and high frequencies during adulthood in a consanguineous Iranian family

Evidence that autosomal recessive nonsyndromic hearing loss-77 (DFNB77) is caused by homozygous mutation in the LOXHD1 gene (613072) on chromosome 18q21.

In situ hybridization detected Loxhd1 expression in the developing mouse inner ear at embryonic days 13.5 and 16, but not in any other tissue. At postnatal day 4, expression was detected in cochlear and vestibular hair cells, with highest concentration in the nucleus. Loxhd1 progressively localized to the cytoplasm, and in the adult, Loxhd1 was expressed in hair cells along the length of stereocilia.

Using an N-ethyl-N-nitrosourea (ENU) mutagenesis screen, Grillet et al. (2009) developed the ‘samba’ mouse line that becomes hearing impaired by 3 weeks of age and deaf by 8 weeks of age. Homozygous samba mice showed no other neurologic or vestibular abnormalities, and heterozygous samba mice appeared completely normal. Stereociliary development was not affected in homozygous samba mice, but hair cell function was perturbed and hair cells eventually degenerated.

Grillet et al. (2009) found that samba was a mutation in the mouse Loxhd1 gene that destabilized the beta-sandwich structure of PLAT domain 10. The mutation did not alter mRNA or protein stability or localization of Loxhd1 protein along the length of stereocilia. However, by postnatal day 21, some hair cells showed morphologic defects with fused stereocilia and membrane ruffling at the apical cell surface. Profound degenerative changes were obvious by postnatal day 90, including hair cell loss and a reduction in spiral ganglion neurons. Grillet et al. (2009) hypothesized that the degeneration of spiral ganglion neurons was likely secondary to perturbations in the function and maintenance of hair cells.

LOXHD1 mutations on chromosome 18 known to cause hearing loss are described in Table 3.

TABLE 3
LOXHD1 MUTATIONS (CHROMOSOME 18)

| # | MUTATION NAME | REFERENCE |
|---|---|---|
| 1 | c.2008C > T | Grillet N, et al. (2009) Mutations |
| | (p.Arg670Ter) | in LOXHD1, an evolutionarily conserved |
| | | stereociliary protein, disrupt hair cell |
| | | function in mice and cause progressive |
| | | hearing loss in humans. Am J Hum Genet. |
| | | 85(3): 328-37. |
| 2 | c.3169C > T | Edvardson S, et al. (2011) A |
| | (p.Arg1057Ter) | deleterious mutation in the LOXHD1 gene |
| | | causes autosomal recessive hearing loss in |
| | | Ashkenazi Jews. Am J Med Genet A. |
| | | 155A(5): 1170-2. |
| | | Grillet N, et al. (2009) Mutations |
| | | in LOXHD1, an evolutionarily conserved |
| | | stereociliary protein, disrupt hair cell |
| | | function in mice and cause progressive |
| | | hearing loss in humans. Am J Hum Genet. |
| | | 85(3): 328-37. |
| 3 | c.2303delG | Edvardson S, et al. (2011) A |
| | (p.Gly768Alafs) | deleterious mutation in the LOXHD1 gene |
| | | causes autosomal recessive hearing loss in |
| | | Ashkenazi Jews. Am J Med Genet A. |
| | | 155A(5): 1170-2. |
| | | Grillet N, et al. (2009) Mutations |
| | | in LOXHD1, an evolutionarily conserved |
| | | stereociliary protein, disrupt hair cell |
| | | function in mice and cause progressive |
| | | hearing loss in humans. Am J Hum Genet. |
| | | 85(3): 328-37. |
| 4 | c.4099G > T | Edvardson S, et al. (2011) A |
| | (p.Glu1367Ter) | deleterious mutation in the LOXHD1 gene |
| | | causes autosomal recessive hearing loss in |
| | | Ashkenazi Jews. Am J Med Genet A. |
| | | 155A(5): 1170-2. |
| | | Grillet N, et al. (2009) Mutations |
| | | in LOXHD1, an evolutionarily conserved |
| | | stereociliary protein, disrupt hair cell |
| | | function in mice and cause progressive |
| | | hearing loss in humans. Am J Hum Genet. |
| | | 85(3): 328-37. |
| 5 | c.2497C > T | Edvardson S, et al. (2011) A |
| | (p.Arg833Ter) | deleterious mutation in the LOXHD1 gene |
| | | causes autosomal recessive hearing loss in |
| | | Ashkenazi Jews. Am J Med Genet A. |
| | | 155A(5): 1170-2. |
| | | Grillet N, et al. (2009) Mutations |
| | | in LOXHD1, an evolutionarily conserved |
| | | stereociliary protein, disrupt hair cell |
| | | function in mice and cause progressive |
| | | hearing loss in humans. Am J Hum Genet. |
| | | 85(3): 328-37. |
| 6 | c.4714C > T | Edvardson S, et al. (2011) A |
| | | deleterious mutation in the LOXHD1 gene |
| | | causes autosomal recessive hearing loss in |
| | | Ashkenazi Jews. Am J Med Genet A. |
| | | 155A(5): 1170-2. |

U.S. Application Publication No. 2013/0095071, incorporated by reference herein in its entirety, describes gene therapy methods for restoring age-related hearing loss using mutated tyrosine adeno-associated viral vectors to deliver the X-linked inhibitor of apoptosis protein (XIAP) to the round window membrane of the inner ear. However, the publication does not contemplate the delivery of a nucleic acid sequence encoding functional TMPRSS3 or LOXHD1 to prevent or delay the onset of or restore hearing loss or deafness caused by genetic mutation of the TMPRSS3 or LOXHD1 gene, as disclosed herein.

Additionally, an important pitfall in the current state of the art for developing clinical gene therapies for hearing disorders is a lack of animal models that mirror human hearing loss. Many of the available mouse models for genetic hearing losses with adult onset in humans present with congenital hearing loss making delivery studies complex. There are few models with onset of genetic hearing loss after development of hearing. Delivery of vectors in neonatal mice results in different transfection patterns than delivery in adult mice (Shu, Tao, Li, et al., 2016). There is a need for novel animal models that can be used to evaluate rescue of hearing using different vector systems and gene targets.

In view of the above, cochlear implantation is one common method of treatment of choice for addressing hearing loss ranging from severe to profound. A cochlear implant is a small, complex electronic device that can help to provide a sense of sound to a person who is profoundly deaf or severely hard-of-hearing. The implant consists of an external portion that sits behind the ear and a second portion that is surgically placed under the skin.

While tremendous advances in cochlear implant design and performance have occurred over the years, there are still patients who do poorly in terms of speech outcomes with implants. Recent studies have demonstrated that mutations in the two genes that cause deafness, TMPRSS3 and LoxHD1, also have poor outcomes in cochlear implant results¹. Specifically, the TMPRSS3 mutant patient has dysfunction of their spiral ganglion². During evaluation of a mouse TMPRSS3 mutant model, it was demonstrated that hair cells degenerated initially and was followed shortly after by the degeneration of spiral ganglion cells³. Permanent damage to the hair cells of the inner ear results in sensorineural hearing loss, leading to communication difficulties in a large percentage of the population. Hair cells are the receptor cells that transduce the acoustic stimulus. Regeneration of damaged hair cells would provide an avenue for the treatment of a condition that currently has no therapies other than prosthetic devices.

During evaluation of human patients with TMPRSS3 mutations, it was demonstrated that cochlear implant function declines with age, which suggests that the delayed degeneration of spiral ganglion cells also occurs in the human population⁴. The foregoing suggests that cochlear implants alone may not be enough to combat hearing loss.

Opportunities, therefore, exist to provide a combination of molecular therapeutics (e.g., gene therapy) for hearing loss in combination with cochlear implantation.

### SUMMARY

Embodiments of the present disclosure relate to, among other things, gene therapy systems and methods useful in treating and/or preventing hearing loss. Systems and methods described herein relate to combination gene therapy with cochlear implantation to repair and/or rescue degenerating hair cells and/or degenerating spiral ganglion cells depending on the time of intervention.

Each of the embodiments disclosed herein may include one or more of the features described in connection with any of the other disclosed embodiments.

Disclosed herein is an expression vector including the nucleic acid sequence of SEQ ID NO:1 or SEQ ID NO:2, or a nucleic acid sequence having at least 90% sequence identity to the nucleic acid of SEQ ID NO:1 or SEQ ID NO:2, wherein the nucleic acid sequence is operatively linked to a promoter. Also disclosed herein is a pharmaceutical composition for use in a method for the treatment or prevention of hearing loss that includes an expression vector having the nucleic acid sequence of SEQ ID NO:1 or SEQ ID NO:2, or a nucleic acid sequence having at least 90% sequence identity to the nucleic acid of SEQ ID NO:1 or SEQ ID NO:2, wherein the nucleic acid sequence is operatively linked to a promoter. In some embodiments, the nucleic acid sequence has at least 75%, at least 80%, at least 85%, at least 90%, at least 95%, at least 96%, at least 97%, at least 98%, or at least 99% sequence identity to the nucleic acid sequence of SEQ ID NO:1 or SEQ ID NO:2. In some embodiments, the expression vector is selected from an adeno-associated viral vector, an adenoviral vector, a herpes simplex viral vector, a vaccinia viral vector, a helper dependent adenoviral vector or a lentiviral vector. In some embodiments, the vector is an adeno-associated viral vector selected from AAV2, AAV2/Anc80, AAV5, AAV6, AAV6.2, AAV7, AAV8, AAV9, AAVrh8, AAVrh10, AAVrh39, AAVrh43AAV1, AAV2, AAV3, AAV4, AAV5, AAV6, AAV7, AAV8, Anc80, or a synthetic version of an adeno associated viral vector serotype. In some embodiments, the adeno-associated viral vector is AAV2, Anc80, or a synthetic version of an adeno associated viral vector serotype. In some embodiments, the promoter is selected from any hair cell promoter that drives the expression of an operably linked nucleic acid at early development and maintains expression throughout the life, for example, TMPRSS3 promoters, human cytomegalovirus (HCMV) promoters, cytomegalovirus/chicken beta-actin (CBA) promoters, Myo7a promoters or Pou4f3 promoters.

Disclosed herein is a cell having an expression vector that includes the nucleic acid sequence of SEQ ID NO:1 or SEQ ID NO:2, or a nucleic acid sequence having at least 90% sequence identity to the nucleic acid of SEQ ID NO:1 or SEQ ID NO:2, wherein the nucleic acid sequence is operatively linked to a promoter. In some embodiments, the nucleic acid sequence has at least 75%, at least 80%, at least 85%, at least 90%, at least 95%, at least 96%, at least 97%, at least 98%, or at least 99% sequence identity to the nucleic acid sequence of SEQ ID NO:1 or SEQ ID NO:2. In some embodiments, the cell is a stem cell. In some embodiments, the stem cell is an induced pluripotent stem cell.

Disclosed herein is a method for treating or preventing hearing loss, including administering to a subject in need thereof an effective amount of an expression vector that includes the nucleic acid sequence of SEQ ID NO:1 or SEQ ID NO:2, or a nucleic acid sequence having at least 90% sequence identity to the nucleic acid of SEQ ID NO:1 or SEQ ID NO:2, wherein the nucleic acid sequence is operatively linked to a promoter. In some embodiments, the nucleic acid sequence has at least 75%, at least 80%, at least 85%, at least 90%, at least 95%, at least 96%, at least 97%, at least 98%, or at least 99% sequence identity to the nucleic acid sequence of SEQ ID NO:1 or SEQ ID NO:2. In some embodiments, the expression vector is selected from an adeno-associated viral vector, an adenoviral vector, a herpes simplex viral vector, a vaccinia viral vector, a helper dependent adenoviral vector or a lentiviral vector. In some embodiments, the vector is an adeno-associated viral vector selected from AAV2, AAV2/Anc80, AAV5, AAV6, AAV6.2, AAV7, AAV8, AAV9, AAVrh8, AAVrh10, AAVrh39, AAVrh43, AAV1, AAV2, AAV3, AAV4, AAV5, AAV6, AAV7, AAV8 or Anc80, or a synthetic version of an adeno associated viral vector serotype. In some embodiments, the adeno-associated viral vector is AAV2, Anc80, or a synthetic version of an adeno associated viral vector serotypes. In some embodiments, the promoter is selected from any hair cell promoter that drives the expression of an operably linked nucleic acid sequence at early development and maintains expression throughout the life, for example, TMPRSS3 promoters, human cytomegalovirus (HCMV) promoters, cytomegalovirus/chicken beta-actin (CBA) promoters, Myo7a promoters or Pou4f3 promoters. In some embodiments, the expression vector is administered into the inner ear of the subject, for example, by injection. In some embodiments, the delivery method is selected from cochleostomy, round window membrane, canalostomy or any combination thereof (see, Erin E. Leary Swan, et al. (2008) Inner Ear Drug Delivery for Auditory Applications. Adv Drug Deliv Rev. 60(15):1583-1599). In some embodiments, the expression vector is delivered into the scala media via the endolymphatic sac (Colletti V, et al. (2010) Evidence of gadolinium distribution from the endolymphatic sac to the endolymphatic compartments of the human inner ear. Audiol Neurootol. 15(6):353-63; Marco Mandalà, MD, et al. (2010) Induced endolymphatic flow from the endolymphatic sac to the cochlea in Ménière's disease. Otolaryngology—Head and Neck Surgery. 143, 673-679; Yamasoba T, et al. (1999) Inner ear transgene expression after adenoviral vector inoculation in the endolymphatic sac. Hum Gene Ther. 10(5):769-74). In some embodiments, the subject has one or more genetic risk factors associated with hearing loss. In some embodiments, one of the genetic risk factors is a mutation in the TMPRSS3 gene. In some embodiments, the mutation in the TMPRSS3 gene is selected from any one or more TMPRSS3 mutations known to cause hearing loss (see, for example, Table 2). In some embodiments, one of the genetic risk factors is a mutation in the LOXHD1 gene. In some embodiments, the mutation in the LOXHD1 gene is selected from any one or more LOXHD1 mutations known to cause hearing loss (see, for example, Table 3). In some embodiments, the subject does not exhibit any clinical indicators of hearing loss.

In some embodiments, an expression vector described herein is administered as a combination therapy with one or more expression vectors comprising other nucleic acid sequences and/or with one or more other active pharmaceutical agents for treating hearing loss. For example, a combination therapy may include a first expression vector that has the nucleic acid sequence of SEQ ID NO:1 and a second expression vector that has the nucleic acid sequence of SEQ ID NO:2, wherein both expression vectors are administered to a subject as part of a combination therapy to treat hearing loss.

Disclosed herein is a transgenic mouse having a human TMPRSS3 gene with a mutation selected from any one or more TMPRSS3 mutation known to cause hearing loss (see, for example, Table 2). Disclosed herein is a transgenic mouse having a human LOXHD1 gene with a mutation selected from any one or more LOXHD1 mutation known to cause hearing loss (see, for example, Table 3).

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 shows a cDNA sequence encoding wild-type human TMPRSS3 (GenBank Accession No. BC074847.2).

FIG. 2 shows the wild-type human TMPRSS3 amino acid sequence encoded by the cDNA in FIG. 1.

FIG. 3 shows a cDNA sequence encoding wild-type human LOXHDI (GenBank Accession No. AK057232.1).

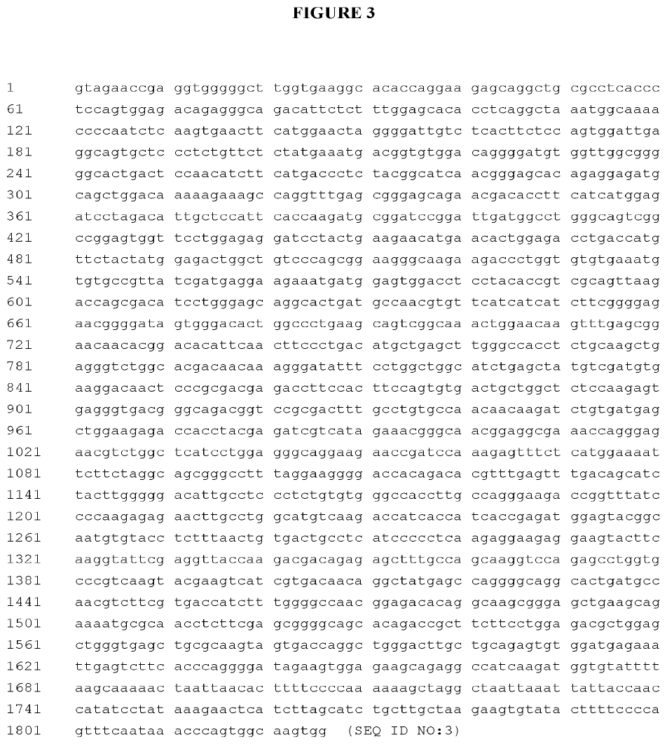

FIG. 4 shows the wild-type human LOXHDI amino acid sequence encoded by the cDNA in FIG. 3.

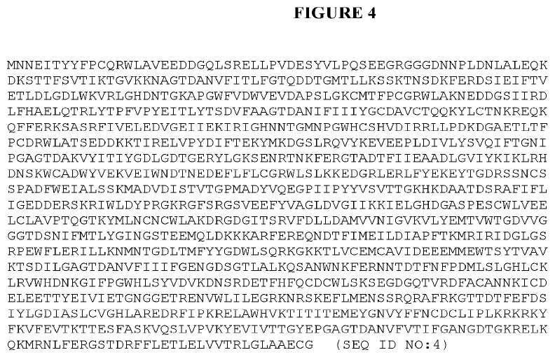

FIG. 5 shows TMPRSS3 immunohistochemistry in the adult mouse cochlea.

FIG. 6 shows an exemplary cochlear implant and the corresponding anatomy of the inner human, according to an aspect of the present disclosure.

FIG. 7 shows an exemplary TMPRSS3 plasmid map beginning at “ORI” and including an initial “AAV2 ITR” vector, a “CMV enhancer”, a “CMV promoter”, a “h-TMPRSS3”, a “bGH poly(A) signal, and a closing “AAV2 ITR” vector.

FIG. 8 illustrates proof of concept by graphically comparing hearing recovery of a disease model mouse receiving gene therapy treatment (treated) vs a disease model mouse not receiving treatment (untreated) by way of Auditory Brainstem Response (ABR) testing.

FIG. 9 illustrates proof of concept by graphically comparing hearing recovery of a disease model mouse receiving gene therapy treatment (treated) vs a disease model mouse not receiving treatment (untreated) by way of Distortion Product Otoacoustic Emissions (DPOAE) testing.

FIG. 10 graphically illustrates proof of concept by graphically comparing auditory neuronal function recovery of a disease model mouse receiving gene therapy treatment (treated) vs a disease model mouse not receiving treatment (untreated) by way of WAVE1 amplitude testing.

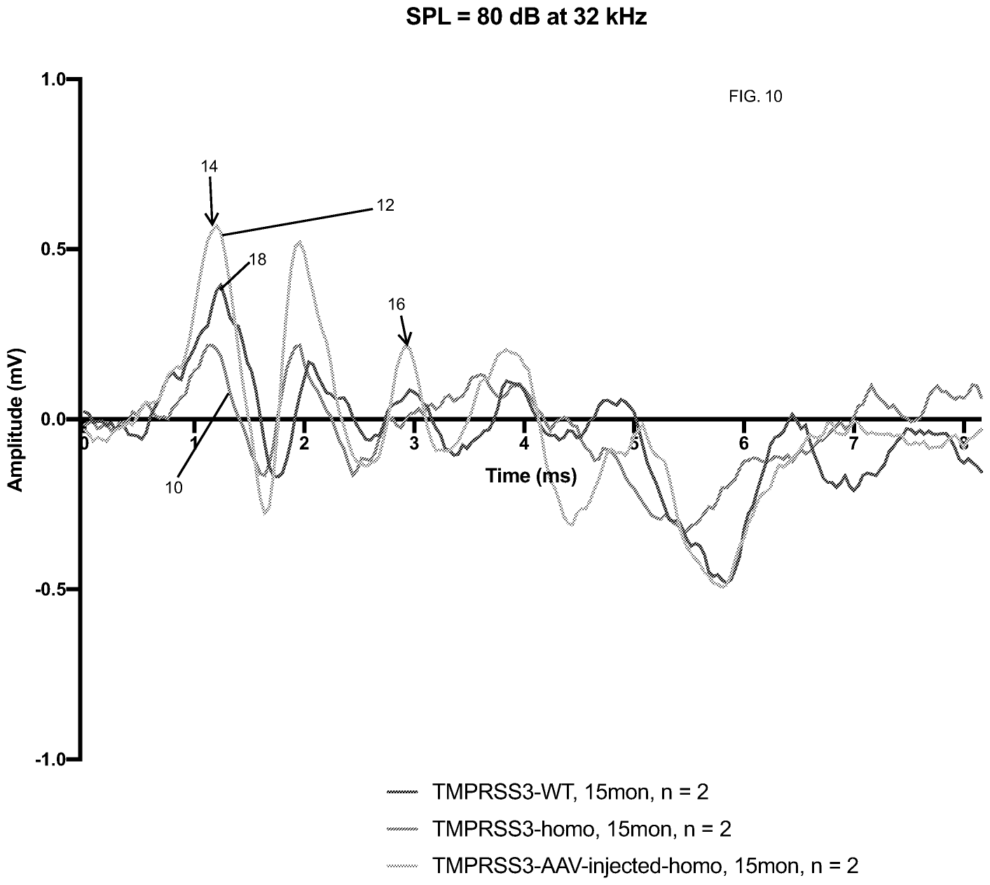

FIG. 11 illustrates the location of the Round Window Membrane (RWM) within the human ear as an exemplary drug delivery site for delivering one or more of the gene therapies taught herein.

### DETAILED DESCRIPTION

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

The present disclosure is drawn to gene therapy systems, and related methods, useful for treating and/or preventing deafness caused by genetic mutation. Examples of two genes that can mutate to cause deafness are the TMPRSS3 gene or the LoxHD1 gene. The systems and methods described herein may utilize a combination of gene therapy (e.g., molecular therapeutics) for hearing loss caused by a genetic mutation together with implantation of a cochlear implant. It can be appreciated that while the systems and methods are in view of gene mutations caused by either the TMPRSS3 gene or the LoxHD1 gene, other gene mutations may be targeted for repair that have been found to cause deafness or hearing loss.

For purposes of the present disclosure, the following definition of “gene therapy” may be used. Gene therapy may refer to when DNA is introduced into a patient to treat a genetic disease. The new DNA usually contains a functioning gene to correct the effects of a disease-causing mutation in the existing gene. Gene transfer, either for experimental or therapeutic purposes, relies upon a vector or vector system to shuttle genetic information into target cells. The vector or vector system is considered the major determinant of efficiency, specificity, host response, pharmacology, and longevity of the gene transfer reaction. Currently, the most efficient and effective way to accomplish gene transfer is using vectors or vector systems based on viruses that have been made replication-defective (PCT Publication No. WO 2015/054653; Methods of Predicting Ancestral Virus Sequences and Uses Thereof).

As used herein, the terms “treat,” “treating,” and “treatment” encompass a variety of activities aimed at desirable changes in clinical outcomes. For example, the term “treat”, as used herein, encompasses any activity aimed at achieving, or that does achieve, a detectable improvement in one or more clinical indicators or symptoms of hearing loss, as described herein.

LOXHD1 gene (for example, as detected in a genetic diagnostic test) but does not yet exhibit clinical indicators or symptoms of hearing loss, thus providing a window during which therapeutic intervention can be initiated. Accordingly, in some embodiments, the present invention provides methods for therapeutic intervention during the period of gradual regression of hearing. The methods of the present invention can be commenced prior to such time period. The methods of treating hearing loss provided by the invention include, but are not limited to, methods for preventing or delaying the onset of hearing loss or the progression of clinical indicators or symptoms of hearing loss.

As used herein, the term “hearing loss” is used to describe the reduced ability to hear sound, and includes deafness and the complete inability to hear sound.

The terms “effective amount” or “therapeutically effective amount,” as used herein, refer to an amount of an active agent as described herein that is sufficient to achieve, or contribute towards achieving, one or more desirable clinical outcomes, such as those described in the “treatment” description above. An appropriate “effective” amount in any individual case may be determined using standard techniques known in the art, such as a dose escalation study. The term “active agent” as used herein refers to a molecule (for example, an AAV vector described herein) that is intended to be used in the compositions and methods described herein and that is intended to be biologically active, for example, for the purpose of treating hearing loss.

The term “pharmaceutical composition” as used herein refers to a composition comprising at least one active agent as described herein or a combination of two or more active agents, and one or more other components suitable for use in pharmaceutical delivery such as carriers, stabilizers, diluents, dispersing agents, suspending agents, thickening agents, excipients, and the like.

The terms “subject” or “patient” as used interchangeably herein encompass mammals, including, but not limited to, humans, non-human primates, rodents (such as rats, mice and guinea pigs), and the like. In some embodiments of the invention, the subject is a human.

As used herein, the terms “vector” or “vectors” may be used. A “vector” may refer to a virus capable of transferring the desired gene into cells, but not capable of taking over or harming cells. To date, adenovirus, adeno-associated virus, herpes simplex virus, vaccinia virus, retrovirus, helper dependent adenovirus and lentivirus have all tested for cochlear gene delivery. Of these, the one that has demonstrated the most potential is adeno associated virus (AAV): it is non-replicating, can efficiently transfer transgenes to the inner ear, and causes no ototoxicity. In particular, AAV can effectively transfect inner hair cells, a critical feature if one hopes to correct genetic defects due to hair cell-specific mutations. To date, a number of different AAV subtypes have been used with success for cochlear gene delivery, demonstrating little if any damage to the organ of Corti. A recent report studying AAV serotypes 1, 2, 5, 6 and 8 demonstrated successful gene expression in hair cells, supporting cells, the auditory nerve and spiral ligament, with hair cells being the most effectively transduced (Lawrence R. Lustig, MD and Omar Akil, PhD (2012) Cochlear Gene Therapy. Curr Opin Neurol. 25(1): 57-60). Examples of AAV vectors that can be administered to the inner ear are further described in U.S. Patent Application No. 2013/0095071, which is incorporated herein by reference in its entirety.

There are currently no approved therapeutic agents for preventing or treating hearing loss or deafness. The current treatment option for those with disabling hearing loss is a cochlear implant. As described herein, by carefully evaluating both the incidence of common recessive causes of hearing loss and taking into account the size of the gene, it is possible to develop a combination treatment therapy system that can be accessible to the common patient population.

Cochlear implants function by bypassing the function of hair cells and directly stimulate spiral ganglion cells. Hair cells are the sensory receptors of both the auditory system and the vestibular system in the ears of all vertebrates. Through mechanotransduction, hair cells detect movement in their environment. However, these cells can deteriorate in certain animals (e.g., humans) because of a mutation in one or more genes (e.g., TMPRSS3, LoxHD1, etc). The spiral (cochlear) ganglion is the group of nerve cells that serve the sense of hearing by sending a representation of sound from the cochlea to the brain. The cell bodies of the spiral ganglion neurons are found in the modiolus, the conical shaped central axis in the cochlea. Therefore, having a functional spiral ganglion is vital for having a cochlear implant function optimally. However, as previously described, these spiral ganglion cells may be susceptible to genetic mutation that result in hearing impairment or hearing loss. Hair cells, as mentioned, may also be susceptible to genetic mutation that may also result in hearing loss or impairment.

According to an aspect of the present disclosure, delivery of a native copy of the TMPRSS3 gene (or any other suitable gene), via a viral vector, may be used to treat either hair cells and/or spiral ganglion cells depending on the vector and the promoters used. Depending on the level of deterioration of the hair cells and/or spiral ganglion cells

Depending on the time of intervention, TMPRSS3 has the potential to rescue degenerating hair cells and/or degenerating spiral ganglion cells. For patients undergoing cochlear implantation because of the degree of hearing loss they have experienced, TMPRSS3 gene therapy may enhance implant function by preserving spiral ganglion function and preventing further degeneration thereby allowing the implant to function optimally given the underlying cellular substrate.

TMPRSS3 is a fairly common cause of hearing loss that is severe enough to warrant cochlear implantation. Additionally, patients with mutations in TMPRSS3 may not respond to cochlear implantation as well as patients with other mutations (Shearer et al., 2017). This presents the opportunity of targeting TMPRSS3, or other genes such as LOXHD1, as a stand-alone therapeutic or in combination with other therapeutic agents and/or cochlear implantation to improve implant outcomes for this disorder. It has been documented that mutations in TMPRSS3 may be the most common cause of postlingual recessive hearing loss that has a fairly limited distribution within the cochlea and, due to the size of the gene, may be built into existing AAV vectors.

U.S. Application Publication No. 2013/0095071, incorporated by reference herein in its entirety, describes gene therapy methods for restoring age-related hearing loss using mutated tyrosine adeno-associated viral vectors to deliver the X-linked inhibitor of apoptosis protein (XIAP) to the round window membrane of the inner ear. However, the publication does not contemplate the delivery of a nucleic acid sequence encoding functional TMPRSS3 or LOXHD1 to prevent or delay the onset of or restore hearing loss or deafness caused by genetic mutation of the TMPRSS3 or LOXHD1 gene, as disclosed herein.

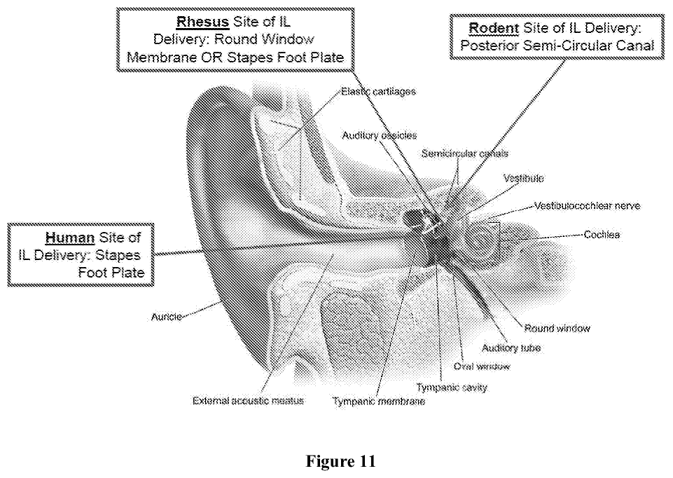

In an exemplary embodiment, and as taught herein, the therapeutic treatment may be delivered through the round window membrane (RMW) of the inner ear using a catheter or port in the cochlear implant, as depicted in FIG. 11. In an exemplary embodiment, the round window membrane (RMW) within the human inner ear may serve as a potential drug delivery site. FIG. 11 is an annotated version of an image of the anatomy of the human ear, available at

https://commons.wikimedia.org/wiki/File:Blausen_0328_EarAnatomy.png. See Blausen.com staff (2014). “Medical gallery of Blausen Medical 2014”. WikiJournal of Medicine 1 (2).

As mentioned above, there are currently no approved therapeutic treatments for preventing or treating hearing loss or deafness and there is a lack of useful preclinical animal models for testing such treatments. The present disclosure therefore describes systems and methods for viral vector gene delivery of TMPRSS3 or LOXHD1 into the inner ear to restore activity of a mutated TMPRSS3 or LOXHD1 gene, promote hair cell survival and restore hearing in patients suffering from hearing loss or deafness, and cell-based and animal-based models for testing such compositions and methods, while also combining treatment with cochlear implantation.

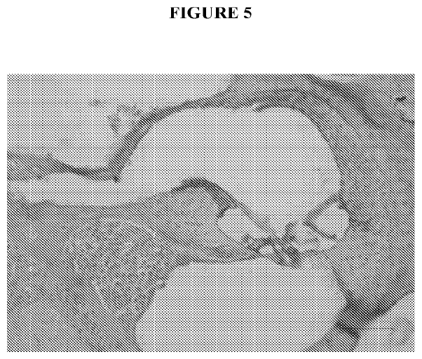

Hearing loss related to mutations in TMPRSS3 (DFNA8/10) can present in a variety of different phenotypes. Both congenital profound hearing loss has been described as well as adult onset progressive hearing losses (Weegerink et al., 2011). Currently, the mechanism by which Tmprss3 dysfunction is unknown. Two mouse models have been developed to date hearing loss at birth and another with onset of hearing loss slightly later time point but still before the maturation of hearing and the mouse. Fasquelle et al. generated an ethyl-nitrosourea-induced mutant mouse carrying a protein-truncating nonsense mutation in Tmprss3. This demonstrated loss of hair cells and degeneration of hearing at post-natal day 12, around the time of maturation of hearing. Additionally saccular hair cells were affected and a delayed degeneration of spiral ganglion cells were noted (Fasquelle et al., 2011). It is unclear from the mouse model whether degeneration of the spiral ganglion is related to degeneration of the organ of Corti or due to dysfunction of Tmprss3 in the spiral ganglion. A number of studies have evaluated the distribution of Tmprrss3 within the mouse inner ear and largely demonstrate presence of Tmprss3 in hair cells and spiral ganglion cells (Fan, Zhu, Li, Ji, & Wang, 2014; Fasquelle et al., 2011). Expression of mouse Tmprss3 was evaluated in 1 month old C57BI5 mice using antibody anti-TMPRSS3 (1:100, ab167160, Abcam, Cambridge, MA). Labelling was seen in inner and outer hair cells, the stria vascularis and in about 50% of spiral ganglion cells (FIG. 5). This suggests that loss of TMPRSS3 function could additionally result in loss of strial function although no changes in endocochlear potential were seen in the Fasquelle mouse model (Fasquelle et al., 2011).

TMPRSS3 genotype-phenotype studies demonstrate a wide range of different forms of hearing loss ranging from profound congenital to adult onset progressive hearing losses (Chung et al., 2014; Gao et al., 2017; Weegerink et al., 2011). Studies suggest that hearing loss due to TMPRSS3 mutations may make up 2 to 5% of patients undergoing adult cochlear implantation (Jolly et al., 2012; Miyagawa, Nishio, & Usami, 2016; Sloan-Heggen et al., 2016). Many of the patients with these mutations have significant amounts of residual hearing. This would make it an attractive target for potential rescue therapy since there would be a substrate of cells that can be treated. There are some divergent studies on the success of cochlear implantation in patients with this mutation. At least some forms of hearing loss induced by loss of TMPRSS3 may not do as well with cochlear implantation than other forms of genetic deafness (Shearer et al., 2017). This is potentially related to the fact that this gene is expressed both in hair cells and in up to 50% of spiral ganglion cells (see FIG. 5). These discrepancies need to be considered when choosing a vector system for delivery. Vectors will be tested with strong hair cell tropism and combined hair cell and spiral ganglion tropism. Differences in vector tropism have also been seen when comparing neonatal and adult inner ear delivery (Shu, Tao, Li, et al., 2016; Shu, Tao, Wang, et al., 2016a). Since the target clinical population are humans with a mature auditory system, disclosed herein is a mouse model that has onset of hearing loss after maturation of hearing in which can be used as a model for both disease progression (see Example 1) and model delivery of rescue therapy to the adult cochlea (see Example 2).

Therefore, an object of the present disclosure is to provide opportunities for using a combination the gene therapy techniques described above together with with cochlear implantation.

### Exemplary Embodiments

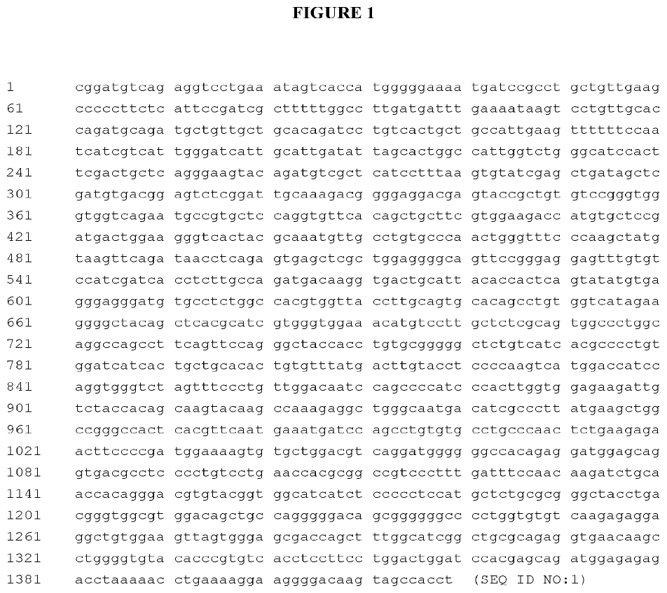

According an exemplary embodiment, the gene therapy techniques taught herein may be delivered in combination with cochlear implantation. In an exemplary embodiment, and with reference to FIG. 1 of the Appendix, a cochlear implant may comprise: 1) a microphone, which may receive sound from the environment; 2) a speech processor, which may select and arrange sounds picked up by the microphone; 3) a transmitter and receiver/stimulator, which may be configured to receive signals from the speech processor and convert them into electric impulses; and 4) an electrode array, which is a group of electrodes that collects the impulses from the stimulator and sends them to different regions of the auditory nerve. In an exemplary embodiment, the cochlear implant may be a small, complex electronic device that can help to provide a sense of sound to a person who is profoundly deaf or severely hard-of-hearing. The implant consists of an external portion that sits behind the ear and a second portion that is surgically placed under the skin.

According to an aspect of the present disclosure, a patient that may qualify for the therapy taught herein can be either: (1) a current user of a cochlear implant or (2) be a candidate for a cochlear implant, but not a current user, i.e. a new cochlear implant user that desires gene therapy treatment in conjunction with a new cochlear implant installation (both done at the same time).

Cochlear implants are designed to mimic the function of a healthy inner ear (or cochlea). They replace the function of damaged sensory hair cells inside the inner ear to help provide clearer sound than what hearing aids can provide. When a person experiences hearing loss or has their hearing impaired significantly, a cochlear implant may be implanted to allow a person to take in external information through their auditory nerve. During sensorineural hearing loss, which means hair cells in a person's inner ear are damaged, the damaged hair cells are no longer capable of sending sounds to their auditory nerve. As alluded to above, a cochlear implant bypasses or skips these damaged hair cells in the inner ear to delivery information directly to the auditory nerve. Studies have shown that certain genes are susceptible to mutation that prematurely damage or deteriorate these hair cells (and/or the spiral ganglion) at birth or sometime later in the person's life. As described above, studies have demonstrated that mutations in the two genes that cause deafness, TMPRSS3 and LoxHD1, may have poor outcomes in cochlear implant results¹. Specifically, the typical TMPRSS3 mutant patient may have dysfunction in either or both of their spiral ganglion and hair cells. During evaluation of a mouse TMPRSS3 mutant model, it was demonstrated that hair cells degenerated initially and was followed shortly after by the degeneration of spiral ganglion cells³. During evaluation of human patients with TMPRSS3 mutations, it was further demonstrated that cochlear implant function declines with age, which suggests that the delayed degeneration of spiral ganglion cells also occurs in the human population⁴.

As stated earlier, patients with mutations in TMPRSS3 may not respond to cochlear implantation as well as patients with other mutations (Shearer et al., 2017). This presents the opportunity of targeting TMPRSS3, or other genes such as LOXHD1, using gene therapy techniques to repair these damaged hair cells and/or spiral ganglion cells in combination with cochlear implantation to improve implant outcomes for this disorder. In other words, the cochlear implant may be used to bypass the defective hair cells and directly stimulate the spiral ganglion cells, and, in combination with the implant, gene therapy may be used to fix the damaged hair cells and/or the spiral ganglion cells that have either been destroyed via natural causes and/or genetic defects. It can be appreciated that any commercially available cochlear implant may be utilized by the systems and methods described herein.

It can be appreciated that in some cases genetic disorders may cause defective hair cells and/or spiral ganglion at the time of birth. In some children, however, the genetic mutation that may result in partial or total hearing loss may come at a later stage in life (e.g., adolescence, adulthood, etc.).

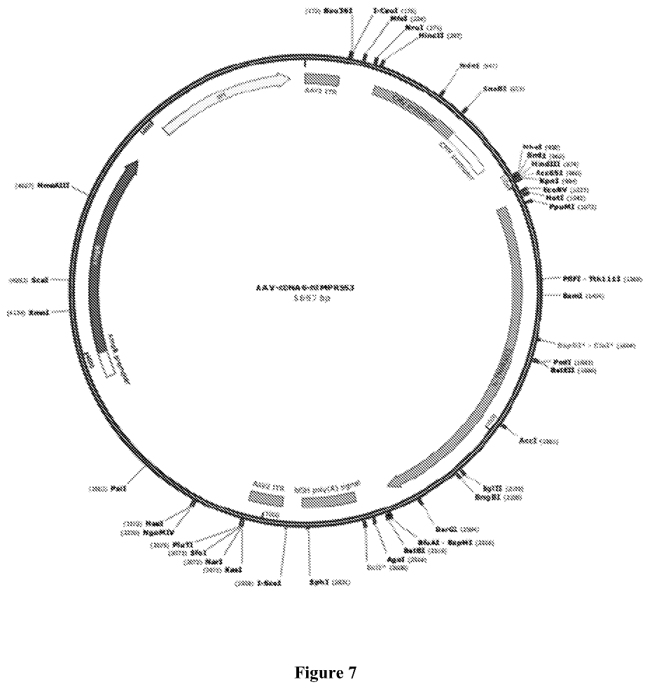

Aspects of the present disclosure cover exemplary embodiments regarding gene therapy (e.g., TMPRSS3, LoxHD1, etc.) for treatment and/or repair of these genetically defective cells of the inner ear (e.g., hair cells, spiral ganglion, etc.). FIG. 7 depicts an exemplary plasmid map for a TMPRSS3 vector construct that may be utilized in gene therapy according to aspects taught herein. The plasmid map illustrates a “AAV-cDNA 6-hTMPRSS3” with 5,667 bp. Cochlear implantation, with gene therapy using the “AAV-cDNA 6-hTMPRSS3” plasmid, may be utilized to achieve one or more of the objectives prescribed in this disclosure.

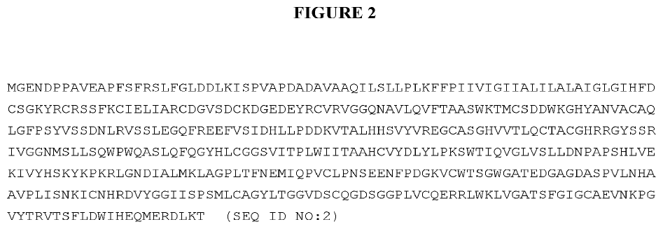

For example, the “AAV-cDNA 6-hTMPRSS3” depicted as FIG. 2 may be used to genetically treat or repair mutations of the TMPRSS3 gene. In doing so, and depending upon the time of the intervention of the gene therapy, the modified TMPRSS3 gene may repair damaged hair cells and/or spiral ganglion caused by mutated and defective genes.

The plasmid map of FIG. 7, in an exemplary embodiment, beginning at “ORI” and including an initial “AAV2 ITR” vector, a “CMV enhancer”, a “CMV promoter”, a “h-TMPRSS3”, a “bGH poly(A) signal, and a closing “AAV2 ITR” vector. Optionally, an additional therapeutic construct “AmpR promoter' may be used. It can be appreciated that other vectors may be utilized to achieve objectives according to aspects of the present disclosure.

### Proof of Concept

MOUSE MODEL: A TRMPSS mouse model in the CBA/J background was generated. These models when bred with the CBA/J strain established the mutant line. The mutation was a knock in model point mutation. The mutation was c.916G>A (p.Ala306Thr) homozygeous mutation.

TMPRSS3 c.916G>A (p.Ala306Thr), has been identified in more than 10 families from Chinese, German, Dutch, and Korean deaf patients, indicating that this mutation is the main contributor to the DFNB8/DFNB10 phenotype in many ethnicities. (Weegerink et al., 2011; J. Lee et al., 2013; J. Chung et al., 2014; M. Elbracht et al., 2007; Gao X et al., 2017)

LAYMAN EXPLANATION OF ABR TEST: The ABR test measures auditory function. The X-axis (Horizontal) lists the Frequencies (Pitch) which are expressed in kilohertz (kh). Numbers to the left of the X-axis are low pitch (like a bass note) as you move to the right, the numbers or pitch get higher (like a flute note). The Y-Axis (Vertical) describes the “Threshold” of hearing or loudness (expressed in decibels or db) i.e. how loud do we have to turn up the volume until the mouse hears.

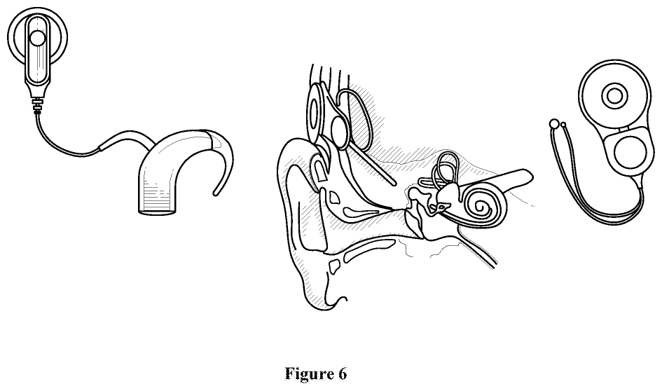

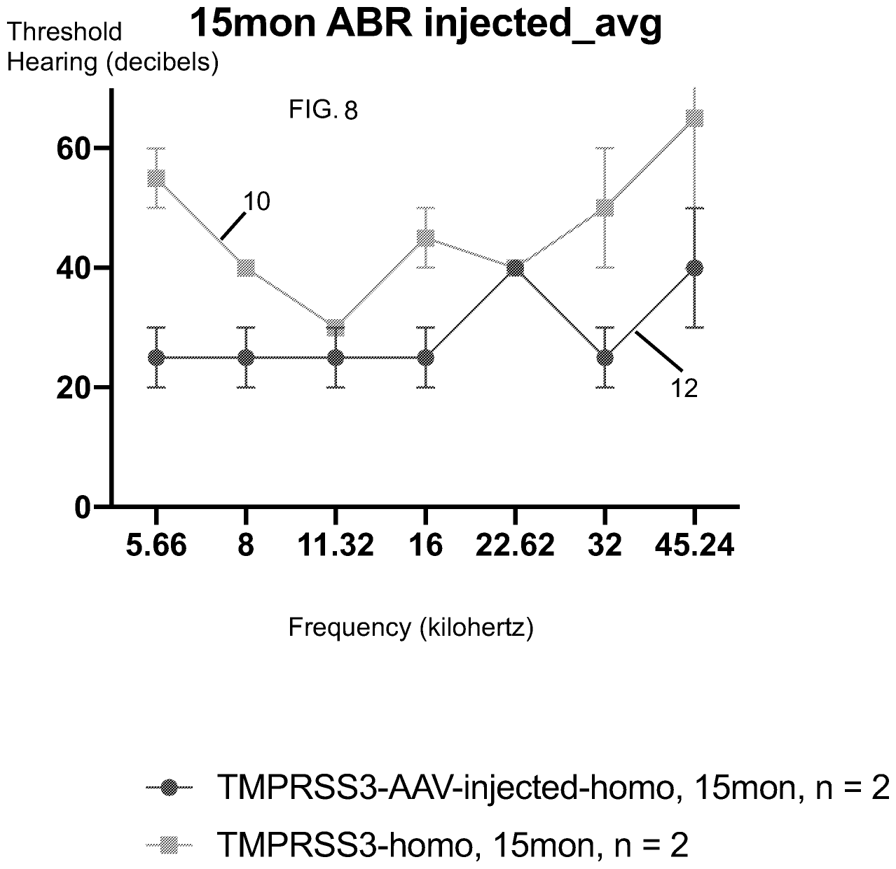

As shown in FIG. 8, the auditory brain response (ABR) test was utilized to measure hearing thresholds at different frequencies for mutant (untreated) mice and mutant experimental (treated) mice. There were 2 mice in the untreated group (10) and 2 mice in the treated group (12). The treated mice (12) had been injected with 1 μL (microliter) of AAV-TMPRSS3 (gene therapy treatment) at the contralateral inner ear. After 1 month (time following injection), the hearing of both treated and un-treated mice were tested using ABR. As shown in FIG. 6, the hearing thresholds for the treated mice (12) were much lower than the hearing thresholds for the control (untreated) mouse (10). Interpretation—The treated mouse (12) hears all frequencies sooner (at a lower volume) than the untreated mouse 10.

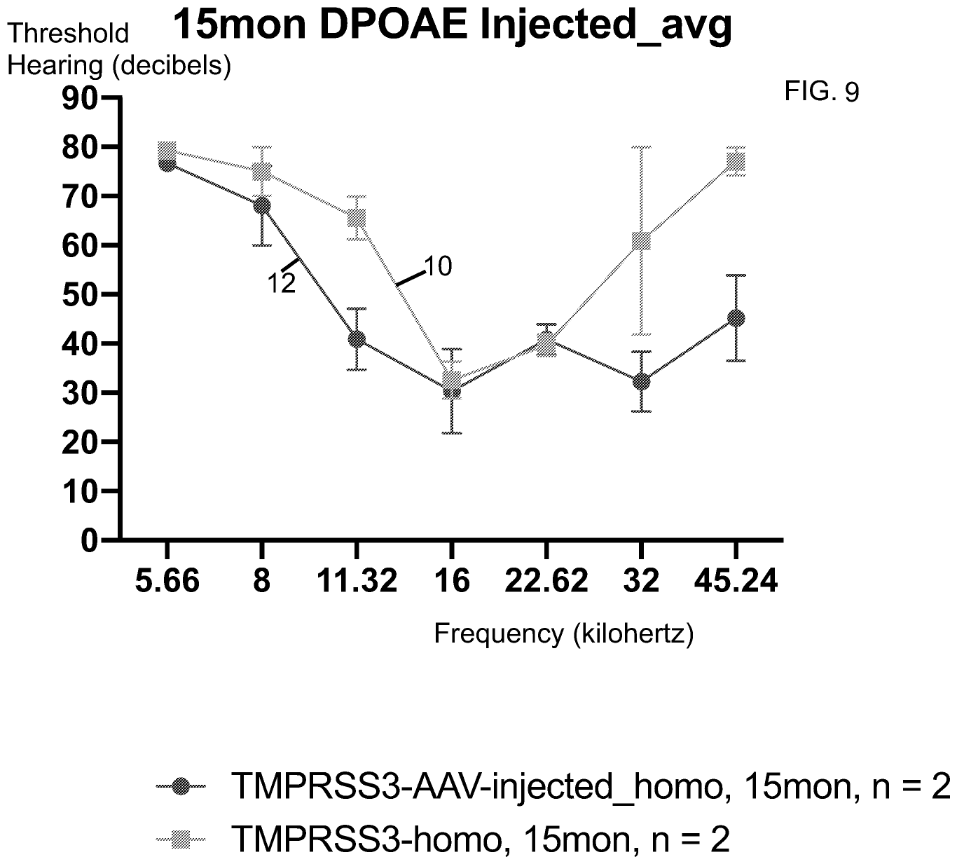

LAYMAN EXPLANATION OF DPOAE TEST: DPOAE is a measure of outer hair cell (OHC) function. The OHCs control volume of incoming sound (i.e. the ear's volume control knob). In FIG. 9, the X and Y axis are same as in FIG. 8. The X-axis is frequency or pitch and Y-axis is threshold or volume needed to hear.

Turning to FIG. 9, shown is a similar improvement utilizing the distortion product otoacoustic emissions test (DPOAE). DPOAE thresholds were elevated in 15 month old untreated mice (10) while the treated mice (12) DPOAE thresholds were restored to normal levels. Interpretation—the treated mouse (12) required less volume to hear the sound than the untreated mouse (10). The data demonstrates that the OHCs of the treated mouse (12) are returning to normal function.

LAYMAN EXPLANATION OF WAVE1 TEST: The WAVE 1 test is an

additional measurement provided by the ABR test. Wave 1 amplitudes measure neuronal activities including the synchronous firing of numerous auditory nerve fibers in the spiral ganglion cells. The (horizontal) X-axis measures the response time to a sound stimulus (click) in milliseconds. The Y-Axis (vertical) describes the “Amplitude” or intensity/sensitivity of the auditory nerve's response to the sound stimulus expressed in millivolts (mv).

With reference to FIG. 8, shown is the auditory evoked potential as a result of acoustic stimulation, measured in millivolts, as a function of time, measured in milliseconds. The acoustic stimulation was at a sound pressure level (SPL) of 80 dB at 32 kHz. The neural response generates a cycle of waves of which the first wave 14 and the third wave 16 are usually considered most significant. In this experiment, WAVE1 amplitudes were measured in treated mice (12) and in untreated mice both homozygous (10) and wild type (18). The WAVE1 amplitudes of the treated mice (12) were significantly greater than the amplitudes for the untreated mice (10 and 18). Interpretation—The treated mice (12) nerve cells are “firing” with greater intensity and sensitivity than untreated mice (10, 18).

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

### REFERENCES

1. Shearer AE, Eppsteiner RW, Frees K, et al. Genetic variants in the peripheral auditory system significantly affect adult cochlear implant performance. Hear Res. 2017; 348:138-142. doi:10.1016/J.HEARES.2017.02.008.
2. Shearer AE, Tejani VD, Brown CJ, et al. In Vivo Electrocochleography in Hybrid Cochlear Implant Users Implicates TMPRSS3 in Spiral Ganglion Function. Sci Rep. 2018; 8(1):14165. doi:10.1038/s41598-018-32630-9.
3. Fasquelle L, Scott HS, Lenoir M, et al. Tmprss3, a transmembrane serine protease deficient in human DFNB8/10 deafness, is critical for cochlear hair cell survival at the onset of hearing. J Biol Chem. 2011; 286(19):17383-17397. doi:10.1074/jbc.M110.190652.
4. Professor Hubert Lowenheim, Personal Communication.
5. N. J. Weegerink, M. Schraders, J. Oostrik et al., “Genotype-phenotype correlation in DFNB8/10 families with TMPRSS3 mutations,” Journal of the Association for Research in Otolaryngology, vol. 12, no. 6, pp. 753-766, 2011.
6. J. Lee, J. I. Baek, J. Y. Choi, U. K. Kim, S. H. Lee, and K. Y. Lee, “Genetic analysis of TMPRSS3 gene in the Korean population with autosomal recessive nonsyndromic hearing loss,” Gene, vol. 532, no. 2, pp. 276-280, 2013.
7. J. Chung, S. M. Park, S. O. Chang et al., “A novel mutation of TMPRSS3 related to milder auditory phenotype in Korean postlingual deafness: a possible future implication for a personalized auditory rehabilitation,” J Mol med (Berl), vol. 92, no. 6, pp. 651-663, 2014.
8. M. Elbracht, J. Senderek, T. Eggermann et al., “Autosomal recessive postlingual hearing loss (DFNB8): compound heterozygosity for two novel TMPRSS3 mutations in German siblings,” Journal of Medical Genetics, vol. 44, no. 6, article e81, 2007.
9. Gao X, Huang SS, Yuan YY, et al., “Identification of TMPRSS3 as a Significant Contributor to Autosomal Recessive Hearing Loss in the Chinese Population,” Journal of Neural Plast. 2017; 2017:3192090. doi: 10.1155/2017/3192090. Epub 2017 Jun. 13.
10. (Weegerink et al., 2011; J. Lee et al., 2013; J. Chung et al., 2014; M. Elbracht et al., 2007; Gao X et al., 2017)

What is claimed is:

1. A system for treating hearing loss, comprising:
a cochlear implant; and
a gene therapeutic construct, wherein the gene therapeutic construct comprises an expression vector for h-TMPRSS3.

2. The system of claim 1, wherein the expression vector further comprises an initial vector, an enhancer, a promoter, a bGH poly(A) signal, and a closing vector.

3. The system of claim 1, wherein the expression vector is an adeno-associated viral vector or a synthetic version of an adeno associated viral vector serotype.

4. The system of claim 3, wherein the expression vector of claim 2, wherein the adeno-associated viral vector is selected from the group consisting of AAV2, AAV2/Anc80, AAV5, AAV6, AAV6.2, AAV7, AAV8, AAV9, AAVrh8, AAVrh10, AAVrh39, AAVrh43, AAV1, AAV2, AAV3, AAV4, AAV5, AAV6, AAV7, AAV8, Anc80, or a synthetic version of an adeno associated viral vector serotype

5. The system of claim 1, wherein the promoter is selected from the group consisting of: TMPRSS3 promoters, Myo7a promoters, human cytomegalovirus (HCMV) promoters, cytomegalovirus/chicken beta-actin (CBA) promoters and Pou4f3 promoters.

6. The system of claim 1, wherein the enhancer is selected from the group consisting of: TMPRSS3 enhancers and human cytomegalovirus (HCMV) enhancers.

7. The system of claim 1, wherein the cochlear implant comprises:
a microphone, the microphone configured to receive sound;
a speech processor, the speech processor configured to select and/or arrange sounds received up by the microphone;
a transmitter and a receiver/stimulator, wherein the transmitter and the receiver/stimulator are configured to receive signals from the speech processor and convert them into electric impulses; and
an electrode.

8. A pharmaceutical composition for use in a method for the treatment or prevention of hearing loss said composition comprising an expression vector comprising an initial vector, an enhancer, a promoter, a bGH poly(A) signal, and a closing vector.

9. The pharmaceutical composition of claim 8, wherein the expression vector is an adeno-associated viral vector or a synthetic version of an adeno associated viral vector serotype.

10. The pharmaceutical composition of claim 9, wherein the adeno-associated viral vector is selected from the group consisting of AAV2, AAV2/Anc80, AAV5, AAV6, AAV6.2, AAV7, AAV8, AAV9, AAVrh8, AAVrh10, AAVrh39, AAVrh43, AAV1, AAV2, AAV3, AAV4, AAV5, AAV6, AAV7, AAV8, Anc80, or a synthetic version of an adeno associated viral vector serotype

11. The pharmaceutical composition of claim 8, wherein the promoter is selected from the group consisting of: TMPRSS3 promoters, Myo7a promoters, human cytomegalovirus (HCMV) promoters, cytomegalovirus/chicken beta-actin (CBA) promoters and Pou4f3 promoters.

12. The pharmaceutical composition of claim 8, wherein the enhancer is selected from the group consisting of: TMPRSS3 enhancers and human cytomegalovirus (HCMV) enhancers.

13. A cell transfected with an expression for the treatment or prevention of hearing loss said expression vector comprising an initial vector, an enhancer, a promoter, a bGH poly(A) signal, and a closing vector.

14. The cell of claim 13, wherein the expression vector is an adeno-associated viral vector or a synthetic version of an adeno associated viral vector serotype

15. The cell of claim 14, wherein the adeno-associated viral vector is selected from the group consisting of AAV2, AAV2/Anc80, AAV5, AAV6, AAV6.2, AAV7, AAV8, AAV9, AAVrh8, AAVrh10, AAVrh39, AAVrh43, AAV1, AAV2, AAV3, AAV4, AAV5, AAV6, AAV7, AAV8, Anc80, or a synthetic version of an adeno associated viral vector serotype

16. The cell of claim 13, wherein the promoter is selected from the group consisting of: TMPRSS3 promoters, Myo7a promoters, human cytomegalovirus (HCMV) promoters, cytomegalovirus/chicken beta-actin (CBA) promoters and Pou4f3 promoters.

17. The cell of claim 13, wherein the cell is a stem cell.

18. The cell of claim 17, wherein the stem cell is an induced pluripotent stem cell.

19. A method for treating or preventing hearing loss in a subject in need thereof, comprising the steps of:
administering to the subject an effective amount of an expression vector comprising an initial vector, an enhancer, a promoter, a bGH poly(A) signal, and a closing vector; and
implanting a cochlear implant in the subject.

20. The method of claim 19, wherein the administration of the expression vector is performed prior to the implantation of the cochlear implant.

21. The method of claim 19, wherein the administration of the expression vector is performed subsequent to the implantation of the cochlear implant.

22. The method of claim 19, wherein the administration of the expression vector and the cochlear implant are performed concurrently.

23. The method of claim 19, wherein the expression vector is an adeno-associated viral vector or a synthetic version of an adeno associated viral vector serotype

24. The method of claim 23, wherein the adeno-associated viral vector is selected from the group consisting of AAV2, AAV2/Anc80, AAV5, AAV6, AAV6.2, AAV7, AAV8, AAV9, AAVrh8, AAVrh10, AAVrh39, AAVrh43, AAV1, AAV2, AAV3, AAV4, AAV5, AAV6, AAV7, AAV8, Anc80 or a synthetic version of an adeno associated viral vector serotype

25. The method of claim 19, wherein the promoter is selected from the group consisting of: TMPRSS3 promoters, Myo7a promoters, human cytomegalovirus (HCMV) promoters, cytomegalovirus/chicken beta-actin (CBA) promoters and Pou4f3 promoters.

26. The method of claim 19, wherein the expression vector is administered by injection into the inner ear of the subject.

27. The method of claim 26, wherein the injection method is selected from the group consisting of cochleostomy, round window membrane, endolymphatic sac, scala media, canalostomy, scala media via the endolymphatic sac, or any combination thereof.

28. The method of claim 19, wherein the subject has one or more genetic risk factors associated with hearing loss.

29. The method of claim 28, wherein one of the genetic risk factors is selected from the group consisting of a mutation in the TMPRSS3 gene or a mutation in the LOXHD1 gene.

30. The method of claim 28, wherein the subject does not exhibit any clinical indicators of hearing loss.


SEQUENCE LISTING (4 sequences)

SEQ ID NO: 1 - DNA, 1419 bp, Homo sapiens
  source 1..1419: mol_type = unassigned DNA
cggatgtcagaggtcctgaaatagtcaccatgggggaaaatgatccgcctgctgttgaagcccccttctcattccgatcgctttttggccttgatgatttgaaaataagtcctgttgcaccagatgcagatgctgttgctgcacagatcctgtcactgctgccattgaagttttttccaatcatcgtcattgggatcattgcattgatattagcactggccattggtctgggcatccacttcgactgctcagggaagtacagatgtcgctcatcctttaagtgtatcgagctgatagctcgatgtgacggagtctcggattgcaaagacggggaggacgagtaccgctgtgtccgggtgggtggtcagaatgccgtgctccaggtgttcacagctgcttcgtggaagaccatgtgctccgatgactggaagggtcactacgcaaatgttgcctgtgcccaactgggtttcccaagctatgtaagttcagataacctcagagtgagctcgctggaggggcagttccgggaggagtttgtgtccatcgatcacctcttgccagatgacaaggtgactgcattacaccactcagtatatgtgagggagggatgtgcctctggccacgtggttaccttgcagtgcacagcctgtggtcatagaaggggctacagctcacgcatcgtgggtggaaacatgtccttgctctcgcagtggccctggcaggccagccttcagttccagggctaccacctgtgcgggggctctgtcatcacgcccctgtggatcatcactgctgcacactgtgtttatgacttgtacctccccaagtcatggaccatccaggtgggtctagtttccctgttggacaatccagccccatcccacttggtggagaagattgtctaccacagcaagtacaagccaaagaggctgggcaatgacatcgcccttatgaagctggccgggccactcacgttcaatgaaatgatccagcctgtgtgcctgcccaactctgaagagaacttccccgatggaaaagtgtgctggacgtcaggatggggggccacagaggatggagcaggtgacgcctcccctgtcctgaaccacgcggccgtccctttgatttccaacaagatctgcaaccacagggacgtgtacggtggcatcatctccccctccatgctctgcgcgggctacctgacgggtggcgtggacagctgccagggggacagcggggggcccctggtgtgtcaagagaggaggctgtggaagttagtgggagcgaccagctttggcatcggctgcgcagaggtgaacaagcctggggtgtacacccgtgtcacctccttcctggactggatccacgagcagatggagagagacctaaaaacctgaaaaggaaggggacaagtagccacct

SEQ ID NO: 2 - protein, 454 aa, Homo sapiens
MGENDPPAVEAPFSFRSLFGLDDLKISPVAPDADAVAAQILSLLPLKFFPIIVIGIIALILALAIGLGIHFDCSGKYRCRSSFKCIELIARCDGVSDCKDGEDEYRCVRVGGQNAVLQVFTAASWKTMCSDDWKGHYANVACAQLGFPSYVSSDNLRVSSLEGQFREEFVSIDHLLPDDKVTALHHSVYVREGCASGHVVTLQCTACGHRRGYSSRIVGGNMSLLSQWPWQASLQFQGYHLCGGSVITPLWIITAAHCVYDLYLPKSWTIQVGLVSLLDNPAPSHLVEKIVYHSKYKPKRLGNDIALMKLAGPLTFNEMIQPVCLPNSEENFPDGKVCWTSGWGATEDGAGDASPVLNHAAVPLISNKICNHRDVYGGIISPSMLCAGYLTGGVDSCQGDSGGPLVCQERRLWKLVGATSFGIGCAEVNKPGVYTRVTSFLDWIHEQMERDLKT

SEQ ID NO: 3 - DNA, 1826 bp, Homo sapiens
  source 1..1826: mol_type = unassigned DNA
gtagaaccgaggtgggggcttggtgaaggcacaccaggaagagcaggctgcgcctcaccctccagtggagacagagggcagacattctctttggagcacacctcaggctaaatggcaaaaccccaatctcaagtgaacttcatggaactaggggattgtctcacttctccagtggattgaggcagtgctccctctgttctctatgaaatgacggtgtggacaggggatgtggttggcgggggcactgactccaacatcttcatgaccctctacggcatcaacgggagcacagaggagatgcagctggacaaaaagaaagccaggtttgagcgggagcagaacgacaccttcatcatggagatcctagacattgctccattcaccaagatgcggatccggattgatggcctgggcagtcggccggagtggttcctggagaggatcctactgaagaacatgaacactggagacctgaccatgttctactatggagactggctgtcccagcggaagggcaagaagaccctggtgtgtgaaatgtgtgccgttatcgatgaggaagaaatgatggagtggacctcctacaccgtcgcagttaagaccagcgacatcctgggagcaggcactgatgccaacgtgttcatcatcatcttcggggagaacggggatagtgggacactggccctgaagcagtcggcaaactggaacaagtttgagcggaacaacacggacacattcaacttccctgacatgctgagcttgggccacctctgcaagctgagggtctggcacgacaacaaagggatatttcctggctggcatctgagctatgtcgatgtgaaggacaactcccgcgacgagaccttccacttccagtgtgactgctggctctccaagagtgagggtgacgggcagacggtccgcgactttgcctgtgccaacaacaagatctgtgatgagctggaagagaccacctacgagatcgtcatagaaacgggcaacggaggcgaaaccagggagaacgtctggctcatcctggagggcaggaagaaccgatccaaagagtttctcatggaaaattcttctaggcagcgggcctttaggaaggggaccacagacacgtttgagtttgacagcatctacttgggggacattgcctccctctgtgtgggccaccttgccagggaagaccggtttatccccaagagagaacttgcctggcatgtcaagaccatcaccatcaccgagatggagtacggcaatgtgtacctctttaactgtgactgcctcatccccctcaagaggaagaggaagtacttcaaggtattcgaggttaccaagacgacagagagctttgccagcaaggtccagagcctggtgcccgtcaagtacgaagtcatcgtgacaacaggctatgagccaggggcaggcactgatgccaacgtcttcgtgaccatctttggggccaacggagacacaggcaagcgggagctgaagcagaaaatgcgcaacctcttcgagcggggcagcacagaccgcttcttcctggagacgctggagctgggtgagctgcgcaagtagtgaccaggctgggacttgctgcagagtgtggatgagaaattgagtcttcacccaggggatagaagtggagaagcagaggccatcaagatggtgtattttaagcaaaaactaattaacacttttccccaaaaaagctaggctaattaaattattaccaaccatatcctataaagaactcatcttagcatctgcttgctaagaagtgtatacttttccccagtttcaataaacccagtggcaagtgg

SEQ ID NO: 4 - protein, 1114 aa, Homo sapiens
MNNEITYYFPCQRWLAVEEDDGQLSRELLPVDESYVLPQSEEGRGGGDNNPLDNLALEQKDKSTTFSVTIKTGVKKNAGTDANVFITLFGTQDDTGMTLLKSSKTNSDKFERDSIEIFTVETLDLGDLWKVRLGHDNTGKAPGWFVDWVEVDAPSLGKCMTFPCGRWLAKNEDDGSIIRDLFHAELQTRLYTPFVPYEITLYTSDVFAAGTDANIFIIIYGCDAVCTQQKYLCTNKREQKQFFERKSASRFIVELEDVGEIIEKIRIGHNNTGMNPGWHCSHVDIRRLLPDKDGAETLTFPCDRWLATSEDDKKTIRELVPYDIFTEKYMKDGSLRQVYKEVEEPLDIVLYSVQIFTGNIPGAGTDAKVYITIYGDLGDTGERYLGKSENRTNKFERGTADTFIIEAADLGVIYKIKLRHDNSKWCADWYVEKVEIWNDTNEDEFLFLCGRWLSLKKEDGRLERLFYEKEYTGDRSSNCSSPADFWEIALSSKMADVDISTVTGPMADYVQEGPIIPYYVSVTTGKHKDAATDSRAFIFLIGEDDERSKRIWLDYPRGKRGFSRGSVEEFYVAGLDVGIIKKIELGHDGASPESCWLVEELCLAVPTQGTKYMLNCNCWLAKDRGDGITSRVFDLLDAMVVNIGVKVLYEMTVWTGDVVGGGTDSNIFMTLYGINGSTEEMQLDKKKARFEREQNDTFIMEILDIAPFTKMRIRIDGLGSRPEWFLERILLKNMNTGDLTMFYYGDWLSQRKGKKTLVCEMCAVIDEEEMMEWTSYTVAVKTSDILGAGTDANVFIIIFGENGDSGTLALKQSANWNKFERNNTDTFNFPDMLSLGHLCKLRVWHDNKGIFPGWHLSYVDVKDNSRDETFHFQCDCWLSKSEGDGQTVRDFACANNKICDELEETTYEIVIETGNGGETRENVWLILEGRKNRSKEFLMENSSRQRAFRKGTTDTFEFDSIYLGDIASLCVGHLAREDRFIPKRELAWHVKTITITEMEYGNVYFFNCDCLIPLKRKRKYFKVFEVTKTTESFASKVQSLVPVKYEVIVTTGYEPGAGTDANVFVTIFGANGDTGKRELKQKMRNLFERGSTDRFFLETLELVVTRLGLAAECG